(19) United States
(12) Patent Application Publication
(10) Pub. No.: US 2015/0196269 A1
(43) Pub. Date: Jul. 16, 2015

(54) SYSTEM AND METHOD FOR REMOTE DETERMINATION OF ACUTE RESPIRATORY INFECTION

(71) Applicant: Xerox Corporation, Norwalk, CT (US)

(72) Inventors: Vaibhav RAJAN, Bangalore (IN); Om Dadaji DESHMUKH, Bangalore (IN); Lalit Keshav MESTHA, Fairport, NY (US); Leslie Edward S., Manipal (IN); Survi KYAL, Rochester, NY (US); Beilei XU, Penfield, NY (US); Nischal Murthy PIRATLA, Fremont, CA (US)

(73) Assignee: Xerox Corporation, Norwalk, CT (US)

(21) Appl. No.: 14/156,108

(22) Filed: Jan. 15, 2014

(51) Int. Cl. A61B 7/00; A61B 7/04

(52) U.S. Cl. CPC A61B 7/003; A61B 7/04

(57) ABSTRACT

What is disclosed is a system and method for determining whether a patient has an acute respiratory infection. In one embodiment, the present method involves using a handheld device to acquire an audio signal of a sound made by a patient coughing. The audio signal is then communicated, by the handheld device, to a remote computing device. Upon receiving the audio signals, signal are repeatedly retrieved from a database of signals associated with different severities of various acute respiratory conditions. A comparison is made between the received audio signal and the retrieved signals. As a result of the comparison, a determination is made whether the patient has an acute respiratory infection. An audio playback device may be employed for playing the audio signal so that a medical professional can listen to that audio signal and facilitate the determination. Various embodiments are disclosed.

### TECHNICAL FIELD

The present invention is directed to a system and method where a patient in a remote location records audio of their breathing/coughing sounds using a handheld device such as a smartphone, and then communicates that audio to a medical profession in another location whereby a preliminary diagnosis is made whether the patient has an acute respiratory infection by a comparison of the patient's audio to known signals contained in a database of signals associated with differing severities of various acute respiratory conditions.

### BACKGROUND

Pneumonia is a major cause of mortality in young children. An acute respiratory infections (ARI) control program developed by the World Health Organization (WHO) has been implemented in various developing countries resulting in a significant decrease in pneumonia related deaths. However, the decrease in mortality is not yet sufficient to reach objectives set forth by the Millennium Developmental Goal, i.e., reduce by two thirds the mortality rate of children 5 years old or younger by the year 2015. Studies conducted by the WHO for Integrated Management of Childhood Illness (IMCI) in developing countries reveal that improvement in case management skills of health-care staff, family and community health practices can have significant impact on health worker performance and their quality of care and the reduction of under-five mortality and improved nutritional status. Their studies also emphasize that implementation of child survival interventions need to be supplemented by activities that strengthen the system support. Large-scale intervention coverage is a needed to achieve these goals. Moreover, the child must be recognized as needing clinical care as promptly as possible. In primary health care facilities, detection and referral of sick children to the required level of medical intervention is essential for further decrease in mortality rate of children with pneumonia. The teachings hereof are in furtherance of this effort.

Accordingly, what is needed in this art is a system and method which can facilitate the determination, by a medical professional, whether a patient in a remote location has an acute respiratory infection.

### INCORPORATED REFERENCES

The following U.S. patents, U.S. patent applications, and Publications are incorporated herein in their entirety by reference.
Breathing Pattern Identification For Respiratory Function Assessment′, U.S. patent application Ser. No. 14/044,043, by Mestha et al.
“Respiratory Physiology: The Essentials”, John B. West, Lippincott Williams & Wilkins; 9^{th} Ed. (2011), ISBN-13: 978-1609136406.

### BRIEF SUMMARY

What is disclosed is a system and method for remotely determining whether a patient has an acute respiratory infection. The sole device required on the patient side is a handheld device, preferably a cellphone with audio/video capture capability and enabled for wireless communication. Patients acquire audio of their breathing/coughing using the microphone built into the device. Audio signals acquired are transmitted by the handheld device to a remote computing device for analysis and preliminary diagnosis of acute respiratory cases with or without the advice of a medical specialist. The present invention leverages prevailing wired and wireless infrastructures which are increasingly reliable in most countries. Embodiments hereof enable infectious respiratory diseases to be identified, treated, tracked and studied.

In one embodiment, a handheld device such as, for example, a smartphone, a tablet, a notebook, and a laptop, is used to acquire an audio signal of a breathing sound of a respiratory patient in a remote location. The acquired audio signal of the patient's breathing sounds is communicated by the handheld device to a remote computing device, i.e., the location of the patient is different (perhaps substantially different) than the location of the computing device. For example, the patient may reside in a village away from a population center while the computing device is in a hospital in a faraway city. If the handheld device is a handheld cellular device then the acquired audio signals are wirelessly communicated to the remote computing device utilizing, for instance, a wireless cellular protocol. The remote computing device receives the acquired audio signal and retrieves, from a database, audio signals that are associated with various acute respiratory conditions. A comparison is then made between the patient's audio signal and the signals retrieved from the database. As a result of the comparison, a determination is made whether the patient has an acute respiratory condition. As used herein, an acute respiratory invention is an upper respiratory tract infection (URI) and/or a lower respiratory tract infection (LRI). The audio signal may be distributed to other medical professionals, secondary and tertiary service providers, governmental agencies, and disease control centers, for further analysis, longitudinal study, data compilation, and the like.

Many features and advantages of the above-described system and method will become readily apparent from the following detailed description and accompanying drawings.

### BRIEF DESCRIPTION OF THE DRAWINGS

The foregoing and other features and advantages of the subject matter disclosed herein will be made apparent from the following detailed description taken in conjunction with the accompanying drawings, in which:

FIG. 1 plots a slice of an example wet cough;

FIG. 2 plots a slice of an example whooping cough;

FIG. 3 plots a slice of an example baby's cry;

FIG. 4 is a front view of one example handheld device capable of utilizing in wireless cellular network;

FIG. 5 is a back view of the handheld device of FIG. 4;

FIG. 6 is a right side view of the handheld device of FIG. 4;

FIG. 7 is a flowchart which illustrates one example embodiment of the present method for determining whether a patient has an acute respiratory infection;

FIG. 8 is a continuation of the flow diagram of FIG. 7 with flow processing continuing with respect to node A; and

FIG. 9 illustrates one example embodiment of a wireless networked system for implementing various aspects of the present method as described with respect to the flow diagrams of FIGS. 7 and 8.

### DETAILED DESCRIPTION

What is disclosed is a system and method for determining whether a patient in a remote location has an acute respiratory infection.

### NON-LIMITING DEFINITIONS

A “patient” refers to a person for which a determination is intended to made whether or not they have an acute respiratory infection. The use of the term “patient” is not intended to limit the scope of the appended claims solely to people who are currently under the care of a physician. Rather, it refers to anyone to be diagnosed for an acute respiratory infection in accordance with the teachings hereof.

An “acute respiratory infection” refers to an upper respiratory tract infection and/or a lower respiratory tract infection, as are generally understood. Such infections may indicate a potentially life threatening respiratory condition such as pneumonia. Generally, an acute respiratory infection is considered pneumonia when the patient has a respiratory rate of either at least 60 bpm and the patient's age is less than 2 months, at least 50 bpm and the patient's age is between 2 to 12 months, and at least 40 bpm and the patient's age is between 12 to 60 months.

An “audio signal” is an electronic recording of the breathing/coughing sound made by the patient. The audio signal is communicated by the handheld device to a remote computing device and is used to determine whether the patient has an acute respiratory infection. The electronic recording can be edited, manipulated, transmitted, received, stored, and played. The audio signal may be processed to identify a breathing pattern for the patient which can help in the determination of the patient's respiratory condition.

A “breathing sound” is a sound of the patient breathing/coughing over a plurality of respiratory cycles.

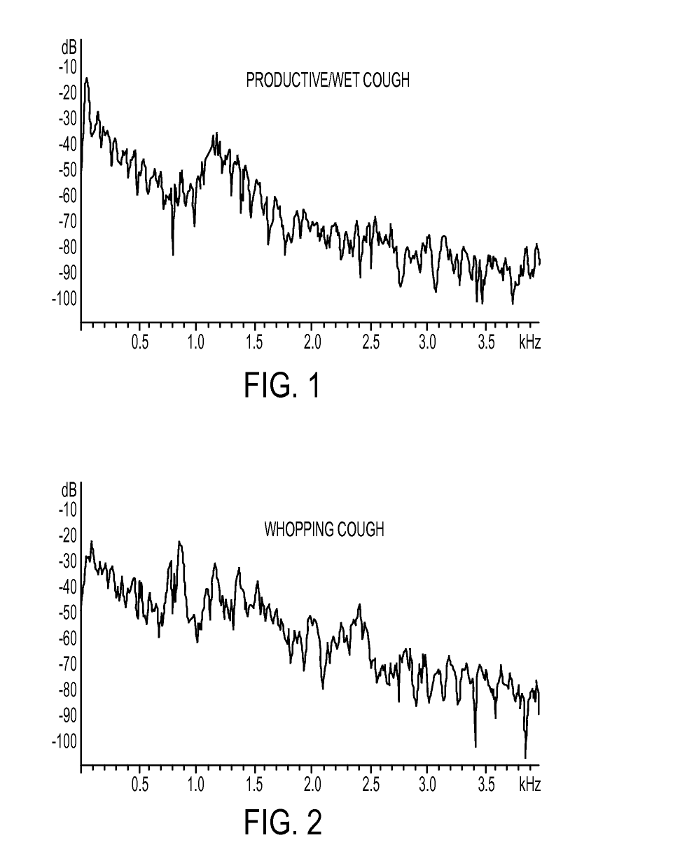

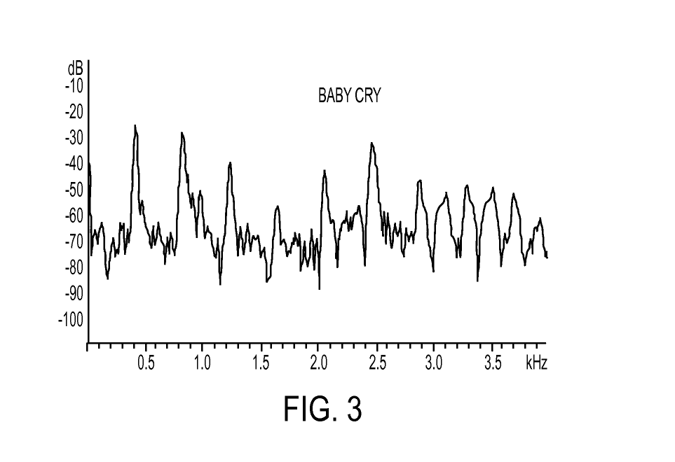

A “cough” (also called “tussis”) is a rapid expulsion of air from the lungs typically in order to clear the lung airways of fluids, mucus, or other matter. Coughing can be voluntary or involuntary. Involuntary coughing is a reflexive event initiated by the lining of the airways. Coughing sounds include: a dry cough (also called a non-productive cough), a wet cough (also called a productive cough), a croup cough (also called a barking cough), a whooping cough (an infectious disease known as Pertussis), a psychogenic cough (also called a habit cough), and a reflex cough caused by having inhaled an irritant of some type from the environment. Each of these coughs is associated with a severity of acute respiratory infection. Different types of cough have different acoustic characteristics. The type of cough most associated with pneumonia is typically a productive/wet cough. Outbound air hits the mucus in the airways and so this type coughing sound typically will have a bulge at lower frequencies. FIG. 1 shows a slice of a wet/productive cough. Coughs don't excite the glottis in a periodic manner, as during the production of vocalic sounds, and hence the spectrum does not exhibit any harmonic structure. This is evident in the slice shown for a whooping cough in FIG. 2. Whereas, the cry signal of FIG. 3 shows harmonic structures. Temporal characteristics of cough signals are also very different across the various types and those of cry signals. For example, the cough signal of FIG. 1 shows very little melody or spectral continuity. The cough signal of FIG. 2 shows abruptness in the temporal onset and offset energy. While the cry signal of FIG. 3 shows short bursts of energy.

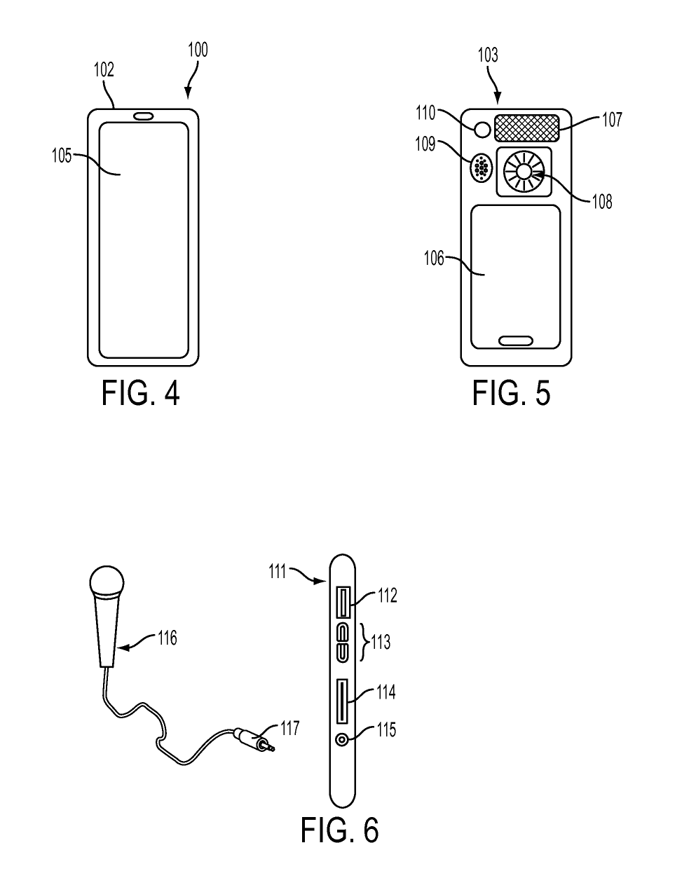

A “handheld device” is a device which is used to acquire an audio signal of the patient's breathing. The handheld device may be, for example, a smartphone, a tablet, a notebook, and a laptop. One such handheld device is shown and discussed with respect to FIGS. 4-6. Such devices typically utilize at least one central processing unit (CPU) also referred to as a “processor”, at least one memory, and at least one storage device which may be a removable device. The handheld device captures audio and, in some embodiments, captures video of the patient. The handheld device is configured to either communicate with various devices using either a wired or wireless protocol. The handheld device may further comprise a display, such as a touchscreen, for displaying user-selectable menu options. The audio signal may be acquired in response to such a selection having been made thereby. The handheld device may be placed in communication with other devices via the world wide web (Internet) and further display a web-based interface whereon a user thereof receives instructions, notifications, alerts, and makes selection thereby. The handheld device is preferably configured to receive a notification and to play or otherwise provide that notification to the user. Such notifications may take the form of an audio message, a text message, an email, a phone call, a video, and an alert. The handheld device may further have a vibration capability. In some embodiments, the audio signal is a streaming audio signal that is acquired and communicated to the remote computing device in real-time.

A “remote computing device” is a device which is used herein to facilitate a determination of the patient's respiratory condition. Example remote computing devices include: an ASIC, a smartphone, a tablet, a notebook, a laptop, a server, a mainframe, a computer workstation, or any combination thereof. In one embodiment, the computing device is configured to communicate back to the handheld device using either a wired or wireless protocol. In one embodiment hereof, a notification is communicated by to patient via the handheld device. The notification may be, for example, an audio message, a text message, an email, a phone call, a video, and an alert signal. Use of the term “remote” means that the computing device is remote from the handheld device, i.e., their locations are different. It can equally be said that the handheld device is remote from the computing device. The handheld device may be used by a patient in a remote village while the computing device is located in a city which has a medical facility or medical offices. Conversely, the handheld device may be used by a patient in the city while the computing device is in a remote village. The two devices may be located in the same village or city, or in different cities, or in different countries. As such, the term “remote computing device” should not be viewed as limiting the scope of the appended claims strictly to computing devices located in remote areas. Various handheld devices available in differing streams of commerce have a female audio-in port which receives a male audio jack. In one embodiment, a microphone is plugged into the audio-in port and used to capture the breathing sounds. In other embodiments, a stethoscope configured to have a male audio jack is plugged into the audio-in port of the handheld device and the stethoscope is used to receive breathing sounds therethrough. In some embodiments, the audio signal is a streaming audio signal that is acquired and communicated to the remote computing device in real-time.

“Communicating the audio signal” means electronically transmitting the audio signal to the computing device. If the handheld device is, for example, a smartphone capable of utilizing wireless cellular communication then the acquired audio signals are transmitted wirelessly over cell-towers. It should be appreciated that the audio signal being communicated by the handheld device to the computing device may utilize both wired and wireless pathways while traversing the distance therebetween.

An “audio playback device”, as is generally understood, is a device which is capable of receiving an audio signal and playing that signal through a speaker so that the signal can be heard by the human auditory system. Although one or both of the handheld device and the computing device may utilize audio playback functionality, the audio playback device may, at least partially, be a separate sound system.

A “database of known signals” that are associated with differing severities of various acute respiratory conditions can be readily generated by digitally recording the breathing sounds of patients who have already been diagnosed with a respiratory condition, and storing those signals in the database. Known signals can be processed, as needed, such that these are made more readily comparable to the audio signals acquired herein using the handheld device. Such processing may include: removing background noise and spurious anomalies, normalization, detrending, amplitude adjustment, frequency and/or phase shifting, linear response compensation (equalization), peak/valley detection, averaging, energy computation, converting to an FFT frequency representation, to name a few. Such methods are well understood in the signal processing arts. Signals or portions thereof may be weighted, as desired. The signals stored in the database are retrieved and compared to the audio signals of the patient. In the training phase, cough signals along with the corresponding ground truth labels (e.g., whether a cough or not and whether pneumonia or not) from health experts would be needed to derive salient acoustic features and/or to select discriminative features. A statistical classifier can then be trained to hypothesize presence or absence of an acute respiratory infection and a classification confidence can be used to estimate the severity of the condition.

“Comparing signals” is intended to be widely construed. The comparison of two signals can be effectuated in a variety ways. For example, two signals can be overlaid and a difference computed. This difference can be compared to a pre-defined threshold to determine whether there is a match therebetween sufficient for medical diagnostic purposes. Specialized circuitry, such as that in an ASIC, can perform quantitative analysis between two input signals and provide, as an output thereof, the differences between the two. For instance, an integral spectrum can be calculated for signal fragments using, for example, a discrete cosine transformation (DCT) and a level of spectrum energy determined and used for comparison purposes. Moreover, other data points such as, for instance, patient age, sex, race, symptoms such as fever, aches, pains, color of phlegm or sputum, persistence, duration, and health vitals, some or all of which have been associated with the audio signals can also be utilized as factors to facilitate a comparison between two audio signals. The method used for comparison will depend on the nature of the system wherein the teachings hereof find their implementation.

### Example Wireless Handheld Device

Reference is now being made to FIGS. 4-6 which illustrate various embodiment of one example handheld device 100 for performing various aspects of the present method.

Handheld device 100 is a mobile cellular device which utilizes in wireless cellular network. FIG. 4 shows a front portion 102 of the wireless handheld device having a touchscreen display 105 capable of displaying a virtual alphanumeric keyboard so that a user thereof can make a selection, enter information or view notifications, results, and the like. The touchscreen display may also be used to display instructions for the user, such as, for instance, a position to hold the device, a time duration to capture video and audio signals, etc. FIG. 5 shows a back portion 103 of the handheld device which includes a battery 106, an audio playback device (speaker 107) for playing audio signals and audio messages, a camera 108 for capturing images and video, a microphone 109 for acquiring audio signals, and an illuminator 110. The handheld device 100 further has a connector (not shown) to connect to a power source such as a charger. FIG. 6 shows a right side portion 111 of the handheld device which includes a USB port 112, a volume control 113, a memory slot 114 for a removable media, and a female port 115 which receives a male jack. In one embodiment, an external microphone 116 has a male audio jack 117 which is plugged into the audio-in port 115 and used thereafter to acquire the audio signals of the patient's breathing sound. In other embodiments, a stethoscope with a male audio jack is plugged into the audio-in port 115 and used to acquire the breathing sounds therethrough. The handheld device also includes non-volatile memory and internal storage for storing variables, formulas, tables, results, applications, software tools, and the like. Any of these may be stored/retrieved from internal storage or non-volatile memory as needed.

It should be appreciated that the handheld device of FIGS. 4-6 is functionally enabled to send/receive email, text, audio, video, a phone call, an alert, and communicate with a remote computing device via a wireless cellular connection. The device may further have a tactile function induced by activating an oscillating cam that introduces a vibration sensation into the chassis of the handheld device. Global Positioning System (GPS) capability is also desirable so that a location of the patient can be determined with specificity, if desired. Some handheld devices have a slideably retractable keyboard in lieu of, or in addition to, a virtual keyboard.

### Example Flow Diagram

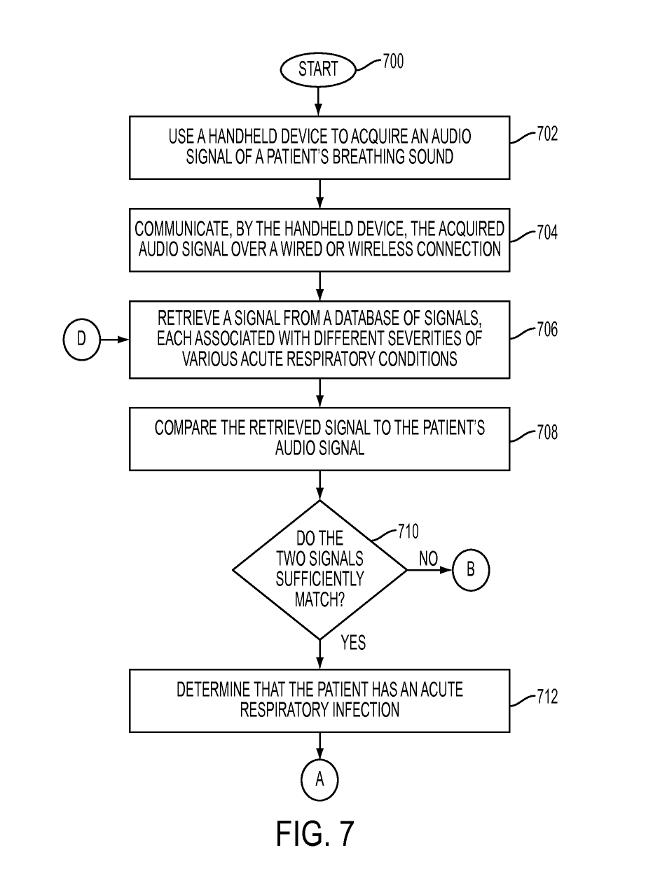

Reference is now made to the flow diagram of FIG. 7 which illustrates one example embodiment of the present method for determining whether a patient has an acute respiratory infection. Flow processing begins at step 700 and immediately proceeds to step 702.

At step 702, use a handheld device to acquire an audio signal of a breathing sound made by a patient. One example handheld device for acquiring an audio signal is shown and discussed with respect to FIGS. 4-6. The audio signal is acquired by the device's microphone functionality. Other embodiments employ an external microphone or specialized stethoscope which is plugged into an audio-in port of the device.

At step 704, communicate the acquired audio signal to a remote computing device over a wired or wireless connection. If the handheld device of FIGS. 4-6 is a smartphone then the acquired audio signal can be communicated using either a wired or wireless communication pathway. Upon receipt of the communicated audio signal, processing continues with respect to step 706.

At step 706, retrieving a next signal from a database of signals associated with different severities of various acute respiratory conditions. On a first iteration hereof, the next signal retrieved is a first signal. On successive iterations, a next signal is retrieved until there are no more signals or until a matching signal has been found.

At step 708, compare the retrieved signal to the received audio signal.

At step 710, a determination is made whether, as a result of the comparison of step 708, there is a match sufficient for medical diagnostic purposes. If so then processing continues with respect to step 712. Otherwise, processing continues with respect to node B.

At step 712, determine that the patient has an acute respiratory infection.

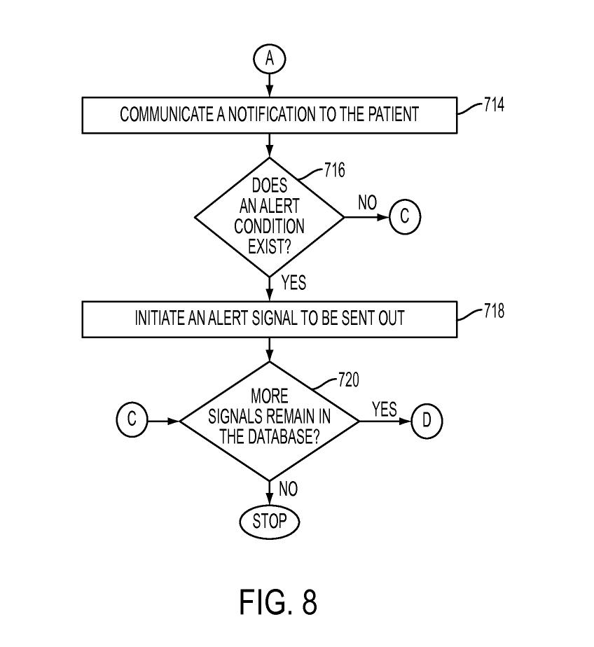

Reference is now being made to the flow diagram of FIG. 8 which is a continuation of the flow diagram of FIG. 7 with flow processing continuing with respect to node A.

At step 714, communicate a notification to the patient. Such a notification can be, for instance, what the patient needs to be doing next in terms of medicine, hospitalization, medical attention, and the like.

At step 716, a determination is made whether an alert condition exists. If so then, at step 718, initiate an alert signal to be sent out. The alert can be sent to, for example, another medical professional, a secondary or tertiary service provider, a government agency, and/or a disease control center. The alert may further include the received audio signal, the matching signal retrieved from the database, along with any other patient information that is deemed appropriate. If it is determined that an alert condition is not appropriate then processing continues with respect to node C.

If, at step 706, the signal retrieved from the database does not sufficiently match the audio signal for medical diagnostic purposes then, at step 720, a determination is made whether more signals remain in the database to be retrieved. If more signals remain in the database then processing continues with respect to node D wherein, at step 706, a next signal is retrieved from the database. This next signal is then compared to the patient's audio signal. A determination is made whether there is a sufficient match therebetween for medical diagnostic purposes. If so then a notification is provided to the patient. A determination is made whether an alert signal should be sent out. Processing repeats in such a manner until either a sufficiently matching signal has been found, or no more signals remain to be retrieved for comparison purposes. Thereafter, further processing stops.

It should be appreciated that the flow diagrams hereof are illustrative. One or more of the operations illustrated in the flow diagrams may be performed in a differing order. Other operations may be added, modified, enhanced, or consolidated. Variations thereof are intended to fall within the scope of the appended claims. Aspects of the flow diagrams will be implemented in hardware executing machine readable program instructions.

### Example Wireless Networked System

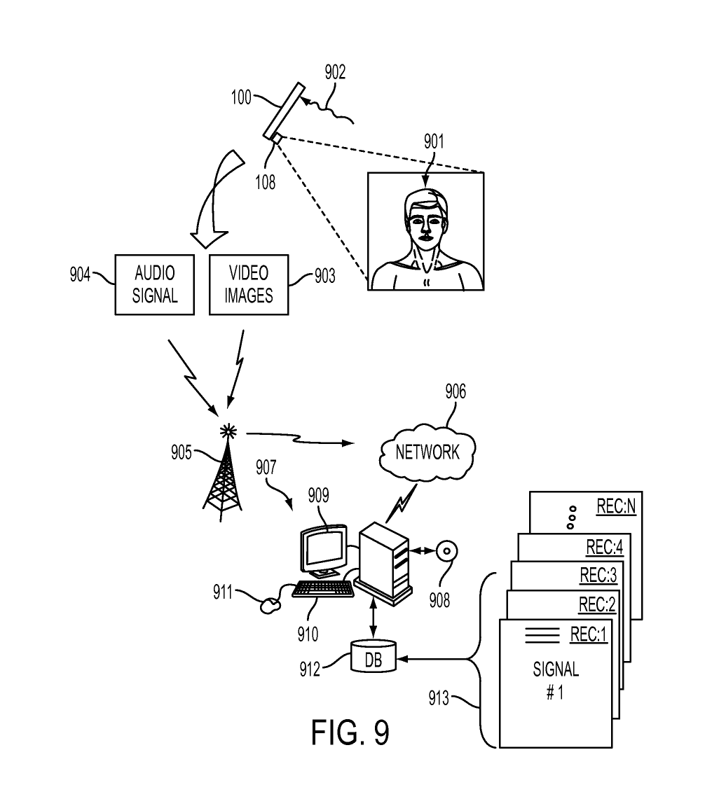

Reference is now being made to FIG. 9 which illustrates one example embodiment of a wireless networked system for implementing various aspects of the present method as described with respect to the flow diagrams of FIGS. 7 and 8. The embodiment shown is illustrative and should not be viewed as limiting the scope of the appended claims strictly to this configuration.

In FIG. 9, handheld device 100 is shown using camera 108 to capture video images of a patient 901 while also acquiring an audio signal of the patient's breathing sound (shown as sound waves 902) using the device's built-in microphone 109. The captured video images 903 of the patient along with the acquired audio signal(s) 904 are wirelessly communicated to a cellular communication tower which transmits the received video and audio signals to a server (not shown) on a network 906. A remote computing device 907, shown in this embodiment as a workstation, is communicatively coupled to the network 906 via wired or wireless pathways. The location of the workstation 907 is considered to be remote from the location of the patient 901. The workstation 907 has a computer case which houses various components such as a motherboard with a processor and memory, a network card, a video card, a hard drive capable of reading/writing to machine readable media 908 such as a floppy disk, optical disk, CD-ROM, DVD, magnetic tape, and the like, and other software and hardware needed to perform the functionality of a computer workstation. The workstation further includes a display device 909, such as a CRT, LCD, or touchscreen device, for displaying patient information, captured video 903, acquired audio signals 904, and the like. A user can view that received information and make selections from menu options displayed on the display 909. Keyboard 910 and mouse 911 effectuate a user input or selection.

The workstation implements a database 912 wherein signals associated with different severities of various acute respiratory conditions and other information are stored in a plurality of records (collectively at 913). As described with respect to steps 706 and 708 of FIG. 7, signals are retrieved from the various records in the database and used for comparison purposes to determine whether a match has been found which is sufficient for medical diagnostic purposes. In other embodiments, the retrieved signals are displayed on a split-screen of the display 909 so that a medical professional can facilitate a determination of the patient's condition. A result of the comparison performed by the remote computing device 907 can also be displayed for review by the medical professional for confirmation before confirming a medical diagnosis. Records stored in a database can be manipulated, updated, and retrieved in response to a query. Information stored on these records, in various embodiments, takes the form of medical histories associated with each of the stored signals which may include information identifying those patients and their treatments and prognoses. A hard drive, internal to the computer case, stores mathematical formulae, functions, and the like, as need for performing a signal comparison so that a match sufficient for medical diagnostic purposes can be determined. Although the database is shown as an external device, the database may be internal to the workstation mounted, for example, on a hard disk therein.

It should be appreciated that the remote computing device 907 has an operating system and other specialized software configured to display alphanumeric values, menus, scroll bars, dials, slideable bars, pull-down options, selectable buttons, and the like, for entering, selecting, modifying, and accepting information needed for processing video images and audio signals in a manner disclosed herein, and for enabling a medical practitioner to make a preliminary medical diagnosis based upon a comparison thereof. In other embodiments, results are communicated to one or more medical practitioners in various locations for their review and input. A practitioner may further communicate instructions back to the user via the handheld device, depending on the implementation. A user or technician may use the user interface of the workstation to set parameters, select image/signal portions for processing. These selections, including the received video images and audio signals, may be stored to storage 908 and 912. Default settings and initial parameters can be retrieved from either the storage devices, as needed.

A medical professional can distribute the video images 903 and/or the audio signals 904 to another medical professional, a tertiary service provider, a government agency, and a disease control center. Various kinds of aggregate summaries can be computed using the patient data. These include, but are not limited to: longitudinal analyses involving statistics of patient cases presented to the present system over a period of time; population-based studies of patients within sub-populations; geography-based studies involving patients in a specified geographical area; socio-economic community-based studies involving patients in a specified social or economic community; and epidemic studies wherein timelines of geographical spread (and severity) of different respiratory infections are formulated and analyzed. Rule based methods can be implemented on the workstation that alert registered government bodies when specific interventions are needed. For example, if the number of infectious cases in a given geography is more than a specified threshold, an alert can be sent out to contain a potential epidemic. Population based and socio-economic statistics sent out by the system can be used to derive healthcare policies by the government agencies. By combining the knowledge of genetic factors from public databases/literature, more sophisticated modeling and analyses can also be performed.

Although shown as a computer workstation, it should be appreciated that the remote computing system can be: an ASIC, a smartphone, a tablet, a notebook, a laptop, a server, a mainframe, and a workstation. The embodiment of the workstation of FIG. 7 is illustrative and may include other functionality known in the arts. Any of the components of the workstation 712 may be placed in communication with the signal processing system 700 or any devices in communication therewith. Any of the modules and processing units of system 700 can be placed in communication with storage device 717 or computer readable media 713 and may store/retrieve therefrom data, variables, records, parameters, functions, and/or machine readable/executable program instructions, as needed to perform their intended functions.

The teachings hereof can be implemented in hardware or software using any known or later developed systems, structures, devices, and/or software by those skilled in the applicable art without undue experimentation from the functional description provided herein with a general knowledge of the relevant arts. One or more aspects of the methods described herein are intended to be incorporated in an article of manufacture. The article of manufacture may be shipped, sold, leased, or otherwise provided separately either alone or as part of a product suite or a service.

It will be appreciated that the above-disclosed and other features and functions, or alternatives thereof, may be desirably combined into other different systems or applications. Presently unforeseen or unanticipated alternatives, modifications, variations, or improvements may become apparent and/or subsequently made by those skilled in this art which are also intended to be encompassed by the following claims. The teachings of any publications referenced herein are each hereby incorporated by reference in their entirety.

What is claimed is:

1. A method for determining whether a patient has an acute respiratory infection, the method comprising:
acquiring, by a handheld device, an audio signal of a sound made by a patient breathing;
communicating, by said handheld device, said acquired audio signal to a remote computing device;
retrieving at least one signal from a database of signals associated with different severities of various acute respiratory conditions;
comparing said retrieved signal to said audio signal; and
determining, as a result of said comparison, whether said patient has an acute respiratory infection.

2. The method of claim 1, wherein said handheld device is any of: a smartphone, a tablet, a notebook, and a laptop.

3. The method of claim 1, wherein said remote computing device is any of: an ASIC, a smartphone, a tablet, a notebook, a laptop, a server, a mainframe, and a computer workstation.

4. The method of claim 1, wherein said acute respiratory invention is any of: an upper respiratory tract infection and a lower respiratory tract infection.

5. The method of claim 1, wherein said breathing sound is any of: a dry cough, a wet cough, a croup cough, a whooping cough, a psychogenic cough, a reflex cough, a grunting sound, a wheezing sound, a crying sound, and a normal respiration sound.

6. The method of claim 1, wherein said audio signal is acquired by any of: a microphone and a stethoscope plugged into an audio-in port of said handheld device, said breathing sounds being acquired therethrough.

7. The method of claim 1, further comprising playing said audio signal on an audio playback device so that a medical professional can listen to that audio signal and facilitate said determination.

8. The method of claim 1, further comprising:
analyzing said audio signal to identify a breathing pattern for said patient, said breathing pattern being one of: Eupnea, Bradypnea, Tachypnea, Hypopnea, Apnea, Kussmaul, Cheyne-Stokes, Biot's, Ataxic, Apneustic, Agonal, and Thoracoabdominal; and
using said identified pattern to facilitate said determination.

9. The method of claim 1, further comprising communicating an instruction to said patient via said handheld device, said instruction comprising any of:
acquiring another audio signal of said patient;
moving said handheld device such that a different audio signal can be acquired;
providing health information;
describing phlegm; and
answering a question.

10. The method of claim 1, further comprising communicating a notification to said patient via said handheld device, said notification comprising any of: an audio message, a text message, an email, a phone call, a video, and an alert signal.

12. The method of claim 1, wherein said audio signal is acquired in response to a user selection having been made using said handheld device, said selection being any of: selecting from a menu displayed on a display screen of said handheld device, selecting from a web-based interface displaying on said display screen, and pressing a button on said handheld device.

13. The method of claim 1, wherein said audio signal comprises a streaming audio signal that is acquired and communicated to said remote computing device in real-time.

14. The method of claim 1, further comprising distributing said audio signal to any of: another medical professional, a tertiary service provider, a government agency, and a disease control center.

15. The method of claim 1, further comprising storing said audio signal to a health record associated with said patient.

16. A system for determining whether a patient has an acute respiratory infection, the system comprising:
a handheld device for acquiring an audio signal of a breathing sound of a patient;
a computing device for receiving said audio signal from said handheld device;
a database of signals associated with different severities of various acute respiratory conditions; and
a processor in communication with a memory and executing machine readable program instructions for performing:
comparing a signal from said database to an audio signal acquired by said handheld device; and
determining, as a result of said comparison, whether said patient has an acute respiratory infection.

17. The system of claim 16, wherein said handheld device is any of: a smartphone, a tablet, a notebook, and a laptop.

18. The system of claim 16, wherein said remote computing device is any of: an ASIC, a smartphone, a tablet, a notebook, a laptop, a server, a mainframe, and a computer workstation.

19. The system of claim 16, wherein said acute respiratory invention is any of: an upper respiratory tract infection and a lower respiratory tract infection.

20. The system of claim 16, wherein said breathing sound is any of: a dry cough, a wet cough, a croup cough, a whooping cough, a psychogenic cough, a reflex cough, a grunting sound, a wheezing sound, a crying sound, and a normal respiration sound.

21. The system of claim 16, further comprising an audio playback device for playing said audio signal so that a medical professional can listen to that audio signal and facilitate said determination.

22. The system of claim 16, further comprising:
analyzing said audio signal to identify a breathing pattern for said patient, said breathing pattern being one of: Eupnea, Bradypnea, Tachypnea, Hypopnea, Apnea, Kussmaul, Cheyne-Stokes, Biot's, Ataxic, Apneustic, Agonal, and Thoracoabdominal; and
using said identified pattern to facilitate said determination.

23. The system of claim 16, further comprising any of: a microphone and a stethoscope, having an audio jack which is plugged into an audio-in port of said handheld device, said breathing sound being acquired therethrough.

24. The system of claim 16, further comprising communicating a notification to said patient via said handheld device, said notification comprising any of: an audio message, a text message, an email, a phone call, a video, and an alert signal.

25. The system of claim 16, further comprising storing said audio signal in a health record associated with said patient.
